(19) United States
(12) Patent Application Publication
(10) Pub. No.: US 2003/0139652 A1
(43) Pub. Date: Jul. 24, 2003

(54) System for pain diagnosis and method therefor

(75) Inventors: Yoon Kyoo Kang, Seoul (KR); Maing Kyu Kang, Seoul (KR)

Correspondence Address: Eugene Mar, BACON & THOMAS, PLLC, Fourth Floor, 625 Slaters Lane, Alexandria, VA, 22314-1176, US

(73) Assignee: ilisoft.co.kr, Seoul (KR)

(21) Appl. No.: 10/216,819

(22) Filed: Aug. 13, 2002

(30) Foreign Application Priority Data
Jan. 22, 2002 (KR) 2002-3611
Jun. 27, 2002 (KR) 2002-36423

(51) Int. Cl. A61B 5/00

(52) U.S. Cl. 600/300

(57) ABSTRACT

Disclosed is a system for pain diagnosis, comprising an input module (10) for receiving inputs of pain areas of a patient onto a three-dimensional human body model, a diagnosis module (20) for deriving diagnosis results, comprising a database (22) for storing data on common pain patterns, a submodule A (21) for checking over a surface of the human body model, comparing the inputted pain areas with respect to the model, and deriving referred pain patterns, a submodule B (23) for allowing the patient to confirm symptoms of the derived pain patterns and assigning weighted values to confirmed pain patterns, a submodule C (24) for comparing images of the confirmed pain patterns with the inputted pain areas, and a submodule D (25) for calculating degrees of matching between the confirmed pain patterns and the inputted pain areas, and an output module (30) for outputting the diagnosis results.

### TECHNICAL FIELD

The present invention relates to a system for diagnosis of pain areas of a patient and a method therefor, and more particularly to a system for pain diagnosis and a method therefor, capable of accurately determining painful sites or pain zones of a patient, thereby facilitating diagnosis and treatment of causes of pain.

### BACKGROUND ART

There are, in general, no standardized methods for expressing pains felt by a patient in a certain body part, and communicating this information between a physician and the patient in a clinic or hospital. For this reason, the patient has a problem upon expressing his/her pain in speech or writing, thus being hard for the physician to accurately recognize a painful site, and further the patient has more difficulties in communicating when suffering multiple pains.

Many experts recommend expressing pains by drawing a picture two dimensionally on a sheet of paper (commonly referred to as a “pain drawing”). However, even when the pain areas are expressed on such a pain drawing in a two dimensional way, it is difficult to accurately determine painful sites and pain zones since the pain areas exist in a human body in three dimensions.

In particular, though specific rules to draw a pain drawing on the paper are provided for the patient, it is difficult to exactly conform thereto. Also, if the pain drawing drawn by the patient deviates from the contour of a human body, its use as diagnosis data is difficult. In addition, most physicians consider such a drawing way to be of little use, limiting its application.

Under these circumstances, there is a need to develop technologies for automatic pain diagnosis, which enables a physician to easily determine patients' pain, and which enables efficient use of a pain drawing for medical examination and as clinical data.

### DISCLOSURE OF THE INVENTION

Therefore, the present invention has been made in view of the above problems associated with conventional methods for pain diagnosis, and the need for efficient and accurate technologies for pain diagnosis, and it is an object of the present invention to provide a system for pain diagnosis, enabling a patient to express painful sites or pain areas to a physician so as to communicate therebetween in efficient and accurate manners, and further enabling the physician to easily derive causes of pain and make a prescription therefor.

In accordance with one aspect of the present invention, the above and other objects can be accomplished by the provision of a system for pain diagnosis, comprising an input module for receiving inputs of pain areas of a patient onto a three-dimensional human body model which comprises uniformly divided multiple cells; a diagnosis module for deriving diagnosis results, comprising a database for storing data on various pain patterns, a submodule A for checking over a surface of the human body model according to divided multiple blocks, comparing the inputted pain areas with respect to the blocks, and deriving referred pain patterns with respect to the blocks corresponding to the patient's pain areas from the database, a submodule B for allowing the patient to confirm symptoms of the derived pain patterns and assigning weighted values to confirmed pain patterns, according to degrees of matchinging upon confirmation, a submodule C for comparing images of the confirmed pain patterns with the inputted pain areas, and a submodule D for calculating degrees of matching between the confirmed pain patterns and the inputted pain areas; and an output module for outputting the diagnosis results derived from the diagnosis module.

The input module may receive inputs of pain symptoms of the patient in a way of answers to questions.

The output module may output the diagnosis results derived from the diagnosis module in a tree structure according to the order of likelihood.

The output module may also represent the pain patterns, which have been shown in the tree structure, on a three-dimensional human body model where the patient's pain areas have been represented.

The output module may also represent nerves, which have been shown in the tree structure, on a three-dimensional human body model where pain areas have been represented.

In addition, the output module may concurrently output symptoms and a prescription corresponding to the diagnosis results.

In accordance with another aspect of the present invention, there is provided a method for pain diagnosis, comprising the steps of: inputting patient's primary information and pain areas in an input module; checking over a surface of a human body model according to divided multiple blocks, comparing the inputted pain areas with respect to the blocks, and deriving corresponding pain patterns with respect to the blocks corresponding to the patient's pain areas from the database, using a submodule A of a diagnosis module; allowing the patient to confirm symptoms of the derived pain patterns and assigning weighted values to confirmed pain patterns, according to degrees of matching upon confirmation, using a submodule B of the diagnosis module; comparing images of the confirmed pain patterns with the inputted pain areas, using a submodule C of the diagnosis module; calculating degrees of matching between the confirmed pain patterns and the inputted pain areas, thereby deriving diagnosis results, using a submodule D of the diagnosis module; and outputting the derived diagnosis results through an output module.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows configuration of a system for pain diagnosis according to the invention;

FIG. 2 is an overall flow chart illustrating a method for pain diagnosis according to the invention;

FIG. 3 is a flow chart illustrating a process performed using a diagnosis-submodule A by a method for pain diagnosis according to the invention;

FIG. 4 is a flow chart illustrating a process performed using a diagnosis-submodule B by a method for pain diagnosis according to the invention;

FIG. 5 is a flow chart illustrating a process performed using a diagnosis-submodule C by a method for pain diagnosis according to the invention;

FIG. 6 is a flow chart illustrating a process performed using a diagnosis-submodule D by a method for pain diagnosis according to the invention;

FIGS. 7 to 13 shows a screen provided by a program for which a system and method for pain diagnosis of the invention has been implemented as a system for diagnosis of myofascial pain syndromes (MPS);

FIG. 7 shows a screen for entering pain areas, by a drawing method, on a three-dimensional human body model implemented in an input module of the system for MPS diagnosis;

FIG. 8 shows a side view of the three-dimensional human body model shown in the right frame of FIG. 7;

FIG. 9 shows a screen where pain areas have been inputted by a drawing method after choosing a square labeled back and waist, among squares shown in the left frame of FIG. 7;

FIG. 10 shows a screen for entering a patient's symptoms, in the format of a questionnaire, which is implemented in an input module of the system for MPS diagnosis;

FIG. 11 shows a screen where diagnosis results have been derived by a diagnosis module after inputting pain areas on a three-dimensional human body model implemented in an input module;

FIG. 12 shows a side view of a drawing represented on a three-dimensional human body model, upon choice of a first pain pattern (that is, Rectus abdominis (lower areas)) among pain patterns listed on the left frame of FIG. 11; and

FIG. 13 shows a screen of a drawing represented on a three-dimensional human body model, upon choice of a second pain pattern (that is, Pyramidalis (L)) among pain patterns listed on the left frame of FIG. 11.

### BEST MODE FOR CARRYING OUT THE INVENTION

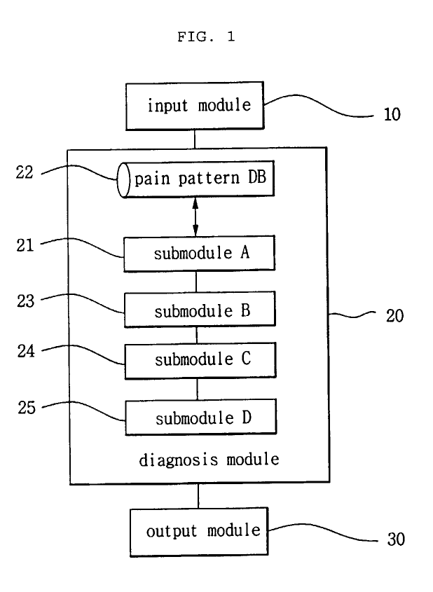

First, a description of a system for pain diagnosis according to the invention is provided referring to FIG. 1.

The system for pain diagnosis of the invention, as can be seen in FIG. 1, comprises an input module 10, diagnosis module 20 and output module 30, as main components for its configuration.

The input module 10 is a component for entering pain areas of a patient by himself/herself or by a physician. The pain areas of the patient are entered onto a three-dimensional human body skin model which comprises uniformly divided cells, after choosing body parts according to sites of the pain areas.

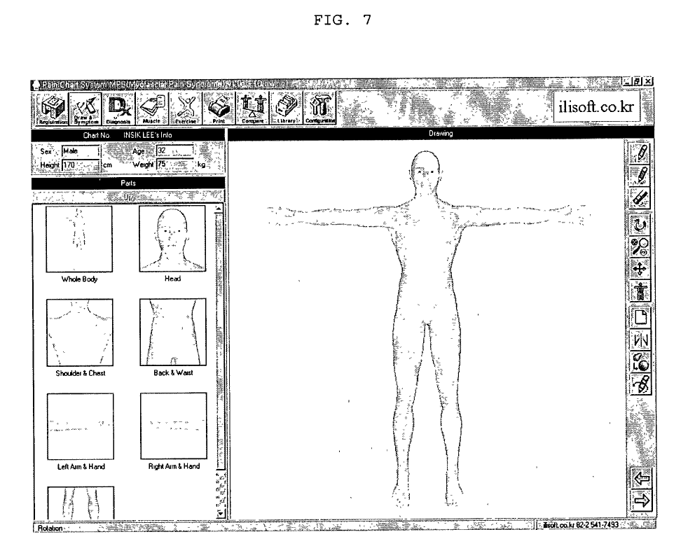

The three-dimensional human body skin model should be sufficiently sophisticated so as to represent all pain areas of the patient, and there should be no regions shielded by other body parts. Thus, the model may be preferably a figure of an adult of 170 cm height, facing the front while spreading both arms horizontally and setting both legs apart at shoulder width, as exemplified in FIG. 7.

Preferably, the input module may allow the accurate input of pain areas by reducing/enlarging, rotating, and translating the body parts, performing editing functions such as deletion and cancellation of execution request, sharpening, and filtering for noise removal, and color-coding according to degrees of pains.

In drawing the painful sites, the most severe and frequent painful sites can be represented in a dark color, while less painful sites can be represented in a light color. In addition, the pains are represented in red, while representing areas with dull sensation and numbness is possible by using a green or blue color, or symbols.

Subdivision into cells on the model is performed according to a Shimada's method, among node generation methods used in finite element analysis. According to the method, based on the assumption that bubbles are packed inside boundary edges of an object, a midpoint of each bubble is considered to be a node of a finite element. Assuming that each bubble exerts an attracting force and repelling force and has a viscosity, the midpoint of the bubble is positioned to ensure that such interacting forces are balanced with respect to entire bubbles. As a result, midpoint coordinates of each bubble are specified to be coordinates of each cell (Kenji Shimada, David C. Gossard; “Automatic Triangular Mesh Generation of Trimmed Parametric Surface for Finite Element Analysis,” Computer Aided Geometric Design, 1: 199-222, 1998).

Since a human body's surface naturally has many bends, it is impossible to subdivide the three-dimensional human body model into completely uniform cells. Accordingly, it is necessary to compensate the Shimada's method. To do this, as a cell correcting tool, Open Graphics Library (Open GL) based on VC++ (Visual C++), the standard for three-dimensional graphics, was used, thus enabling creation of a three-dimensional human body model with a height of 170 cm, a surface area of 15,302 cm², a cell number of 70,370, and an average cell area of 0.2117387 cm².

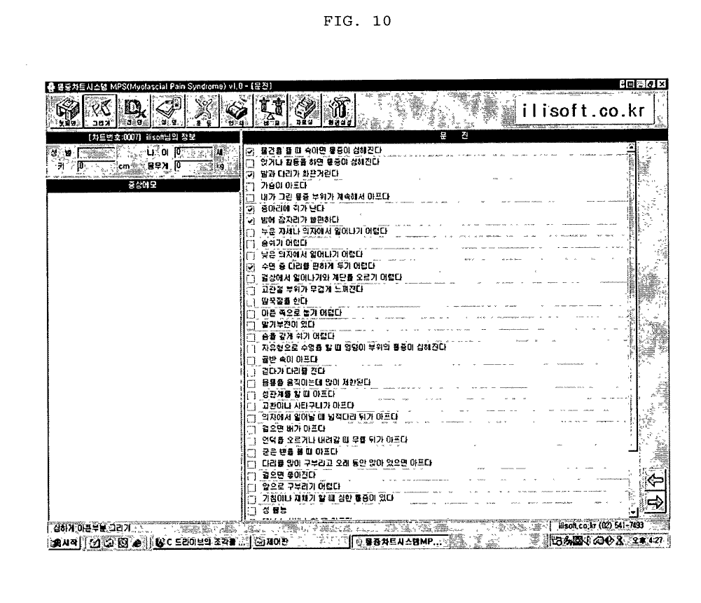

Preferably, the input module 10 of the invention may allow the patient to enter “Yes/No” answers to questions provided in a format of a questionnaire about varying symptoms which may be accompanied by pains, as shown in FIG. 10, as well as pains being represented by a drawing method.

Also preferably, the input module 10 allows a dialog box-based user interface to be implemented. A variety of input ways including a touch screen, or pen mouse using LCD, including a mouse, may be applicable for the patient to easily enter his/her own pain areas.

Meanwhile, the diagnosis module 20 comprises a database of pain patterns 22 and four submodules, that is, submodules A, B, C, and D 21, 22, 23, and 24.

The database of pain patterns 22 stores information about 338 common pain patterns. Pain areas, symptoms and other factors may influence a diagnosis. Among these, the pain areas are of greatest importance, so the other factors can be ignored, because of not substantially affecting a diagnosis. Those pain patterns include a severe pain zone, EPZ (Essential Pain Zone), a less severe zone, SPZ (Spillover Pain Zone), a particular site of a referred muscle which is a cause of pain (TrP; Trigger Point; pain triggering point), and other referred symptoms.

The diagnosis module 20 compares and analyzes the patient's painful sites and areas inputted through the input module 10 versus the common pain patterns, under considerations of the patient's pain areas and symptoms, thereby finding the most similar pain patterns to the patient's pain areas.

The submodules of the diagnosis module 20 are as follows.

The submodule A 21 checks over a surface of the human body model which is divided into 60 blocks, compares the blocks with respect to the pain areas entered by the patient, and derives candidate referred pain patterns with respect to the blocks corresponding to the patient's pain areas from the database,

The submodule B 23 asks the patient about symptoms of the candidate pain patterns and assigns weighted values to confirmed pain patterns, according to degrees of matching.

The submodule C 24 compares images of the confirmed pain patterns with the pain areas entered by the patient, using the Hausdorff distance. The Hausdorff distance is used to compare two images. It refers to the maximum distance of a certain point within one image to the nearest point in the other image (Daniel P. Huttenlocher, Gorgory A. Klanderman, and William J. Rucjlidge; “Comparing Images Using the Hausdorff Distance,” IEEE Trans. on Pattern Analysis and Machine Intelligence, 9(15): 850-863, 1993). Hausdorff distance h (A, B) can be defined as Equation 1 below, where A and B are two respective pain patterns to be compared, a and b are cells of the respective pain patterns, and d(a, b) is a distance between a and b.

$$h ( A , B ) = {max}_{a ε A} \{ {min}_{b ε B} \{ d ( a , b ) \} \} \qquad [ Equation 1 ]$$

The submodule D 25 compares degrees of overlap between the pain areas entered by the patient and the pain patterns. Regions of the pain areas entered by the patient and the pain patterns are respectively divided into SPZ and EPZ. Thus, total 4 overlapping regions are generated. Degrees of matching are calculated by assigning weighted values to the respective overlapping regions. Greater weighted values are assigned to the regions where two EPZs overlap.

The resultant value generated by the submodule D is defined as Equation 2 below.

$$Resultant value = α ( \frac{{E}_{PT} ⋂ {E}_{PA}}{{E}_{PT}} ) + β ( \frac{{E}_{PT} ⋂ {S}_{PA}}{{E}_{PT}} ) + γ ( a {S}_{PT} ⋂ \frac{{S}_{PA}}{{S}_{PT}} + b {S}_{PA} ⋂ \frac{( {S}_{PA} ⋃ {S}_{PA} )}{{S}_{PT}} ) \qquad [ Equation 2 ]$$

provided that α+β+γ=1, a+b=1

wherein, E_{PT} is EPZ of a pain pattern, E_{PA} is EPZ of a patient's pain area entered by the patient, S_{PA} is SPZ of a patient's pain area entered by the patient and S_{PT} is SPZ of a pain pattern.

In combining the parameters α, β, γ, a, and b values used in the submodule D, and the values obtained from the respective modules, it is necessary to correct the weighted values assigned according to modules through experiments and verification conducted by collecting much clinical data.

The output module 30 outputs diagnosis results derived from the diagnosis module 20. The diagnosis results are represented in a tree structure according to the order of likelihood. Each node of the tree structure contains pain patterns. With respect to each pain pattern, an area of overlap between the resultant value obtained from the diagnosis module and the pain areas entered by the patient, is represented.

Upon clicking each pain pattern, referred muscles, a detailed description of the pain pattern, and prescription therefor are presented through windows. On the three-dimensional human body skin model are represented overlapped regions of the pain areas entered by the patient and the pain patterns, and TrPs, thereby direct checking being possible.

The contents of diagnosis results outputted are summarized as follows.

First, the pain patterns similar to the diagnosis result are represented in a tree structure according to the order of likelihood.

Second, the pain patterns shown in the tree structure are represented on the pain areas of the patient.

Third, the nerves shown in the tree structure are represented on the pain areas of the patient.

Fourth, upon showing the pain patterns, referred muscles and TrPs are shown in a drawing, while representing both symptoms and prescription simultaneously.

Fifth, the results are outputted in a file format so as to facilitate a smooth communication between the physician and the patient.

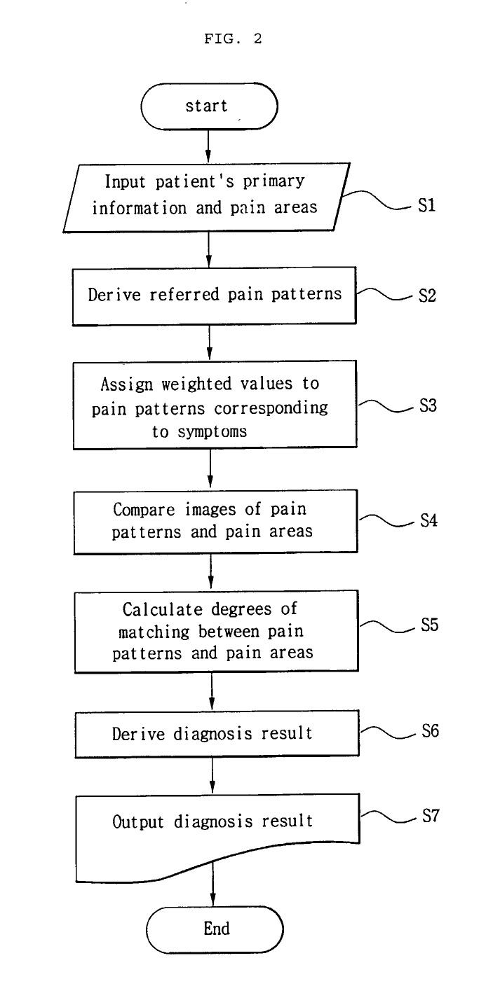

A method for pain diagnosis conducted on the basis of the foregoing system for pain diagnosis of the invention, comprising the steps S1 through S7, is described referring to FIG. 2.

First of all, using an input module 10, primary information including a serial number for the patient, name, age, gender, height, and weight, and pain areas is entered by either a physician or a patient (S1).

Then, using a submodule A 21 of a diagnosis module 20, a surface of a human body is checked according to divided multiple blocks, and the inputted pain areas are compared with respect to the blocks, based on information of pain patterns stored in a pain pattern database 22, thereby deriving referred pain patterns with respect to the blocks corresponding to the patient's pain areas from the pain pattern database (S2).

Using a submodule B 23 of the diagnosis module 20, symptoms of the derived referred pain patterns are confirmed by the patient, and weighted values are assigned to the confirmed pain patterns, according to degrees of matching upon confirmation (S3). Using a submodule C 24 of the diagnosis module 20, the two images of the confirmed pain patterns and the pain areas entered by the patient are compared (S4).

Using a submodule D 25 of the diagnosis module 20, degrees of matching between the confirmed pain patterns and the pain areas entered by the patient are calculated, thereby deriving diagnosis results (S5 and S6).

Finally, using an output module 30, the derived diagnosis results are outputted (S7).

More particular procedures for performing the steps above are described below according to respective submodules.

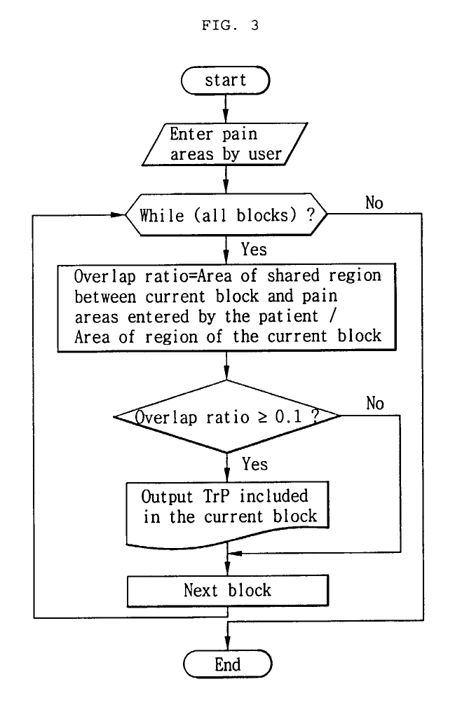

Referring to FIG. 3, as information on pain areas is entered by a user, for example, a patient, the submodule A 21 checks over the surface of a human body model according to divided blocks, and determines whether “an overlap ratio” is 0.1 or more, provided that a value of “a shared area of a region of a current block and a region of pain areas entered by the patient” divided by “an area of the region of the current block” is defined as “the overlap ratio”. When the overlap ratio is 0.1 or more, a candidate TrP included in the block is inputted. Such a TrP serves as data to derive referred pain patterns of the patient from the pain pattern database 22.

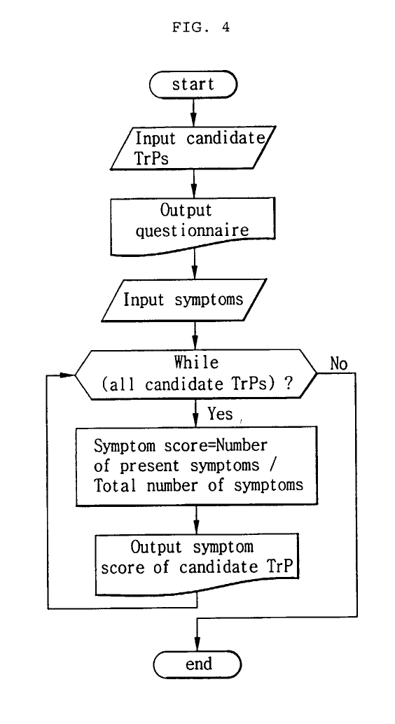

Referring to FIG. 4, as candidate TrPs of the referred pain patterns are inputted, the submodule B 23 outputs question items using the output module 30 so the patient enters symptoms with respect to the question items corresponding to the derived pain patterns, thereby gathering information in a format of questionnaire. When information on all candidate TrPs is ready, “a symptom score” is outputted, provided that a value of “number of present symptoms” divided by “total number of symptoms” is defined as “the symptom score”. Such symptom scores serve as weighted values assigned to the derived pain patterns.

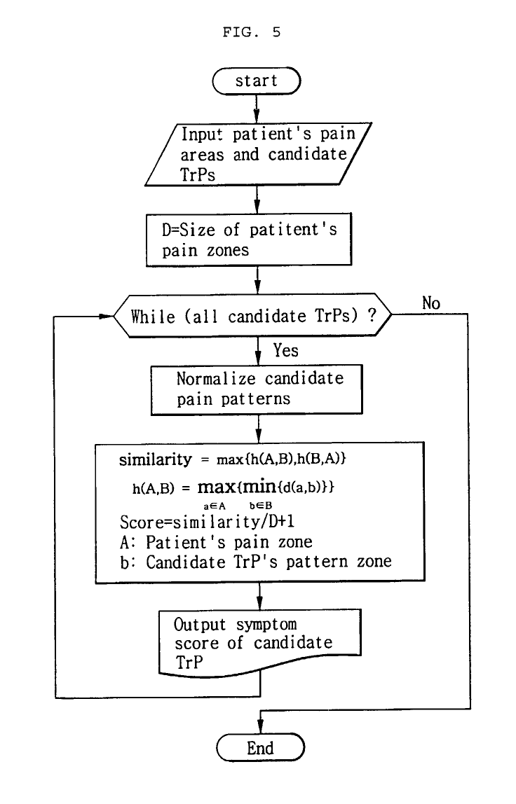

Referring to FIG. 5, once the information on both patient's pain and the candidate TrPs are inputted, the submodule C 24 determines a “D” value, provided that “a size of a patient's pain zone” is defined as a factor “D”. Meanwhile, upon information on all candidate TrPs being prepared, the submodule C normalizes the candidate pain patterns and calculates a similarity between the patient's pain to actual symptoms of a disease and a score corresponding thereto. The submodule C then outputs the scores of the candidate TrPs, and compares images of regions of the pain patterns with those of the inputted pain areas.

The similarity and score are calculated based on Equation 3 below.

$$Similarity = max \{ h ( A , B ) , h ( B , A ) \} h ( A , B ) = {max}_{a ε A} \{ {min}_{b ε B} \{ d ( a , b ) \} \} \qquad [ Equation 3 ]$$

Score=similarity/D+1

where A is one set of two sets for image comparison and B is the other set, and h(A,B) represents the directed “Hausdorff” distance from A to B.

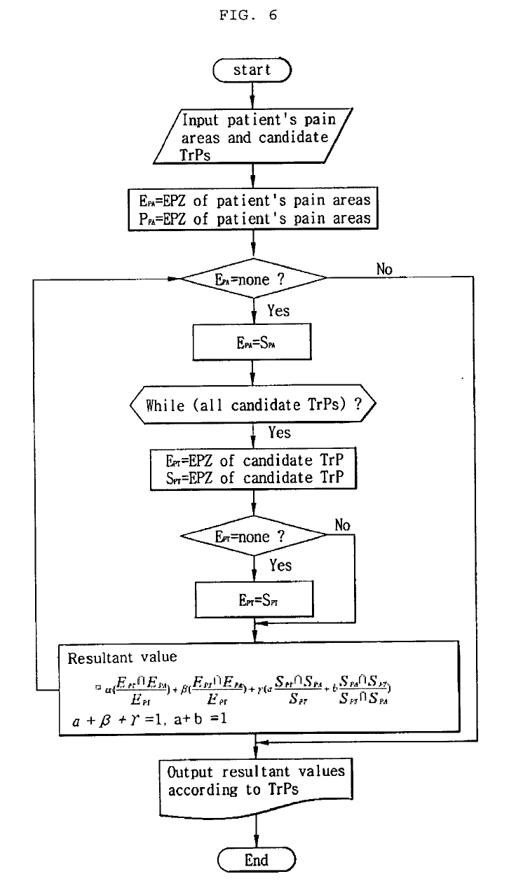

Referring to FIG. 6, once the information on both patient's pain and the candidate TrPs are inputted, the submodule D 25 determines presence or absence of EPZ of the patient's pain (EPA; EPZ of Patient). If E_{PA} is absent, E_{PA} is recognized as SPZ of the patient's pain (S_{PA}; SPZ of Patient). Meanwhile, upon the information on all candidate TrPs being prepared, the submodule D determines presence or absence of EPZ of a candidate TrP (EPT; EPZ of Pattern). If E_{PT} is absent, E_{PT} is recognized as SPZ of the candidate TrP (S_{PA}; SPZ of Patient). Accordingly, a resultant value is calculated. Resultant values according to TrPs are outputted as values of diagnosis results.

At this time, if EPZ of the candidate TrP (E_{PT}) is present, a resultant value is calculated according to this case. The resultant values according to TrPs are outputted. Here, the above resultant values are calculated based on Equation 4 below.

$$Resultant value = α ( \frac{{E}_{PT} ⋂ {E}_{PA}}{{E}_{PT}} ) + β ( \frac{{E}_{PT} ⋂ {E}_{PA}}{{E}_{PT}} ) + γ ( a {S}_{PT} ⋂ \frac{{S}_{PA}}{{S}_{PT}} + b {S}_{PA} ⋂ \frac{{S}_{PT}}{{S}_{PT} ⋂ {S}_{PA}} ) \qquad [ Equation 4 ]$$

provided that α+β+γ=1, a+β=1

wherein, E_{PT}=EPZ entered by the pain patterns, E_{PA}=SPZ entered by the pain patterns, S_{PA}=SPZ entered by the patient, S_{PT}=EPZ entered by the pain patterns.

The foregoing system and method for pain diagnosis according to the invention can be implemented as systems for diagnosis of systemic pain conditions such as fibromyalgia, local pain conditions such as headache, dental pain and chest pain, skin diseases, and burns, as well as myofascial pain syndromes (MPS). Implementation as a diagnosis system to perform acupuncture used in herbal therapy is also possible.

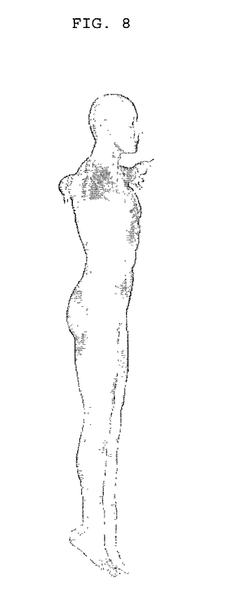

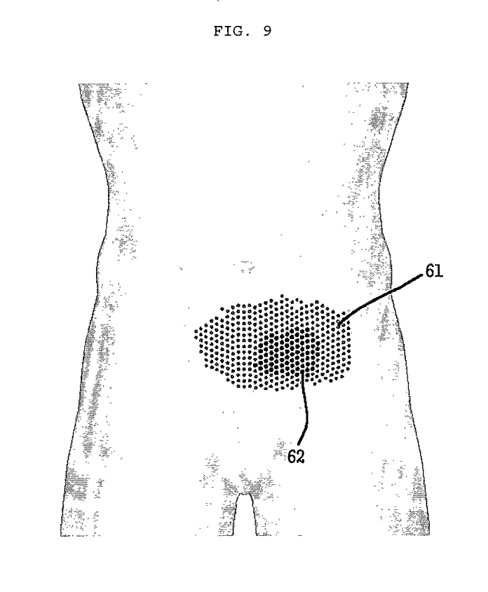

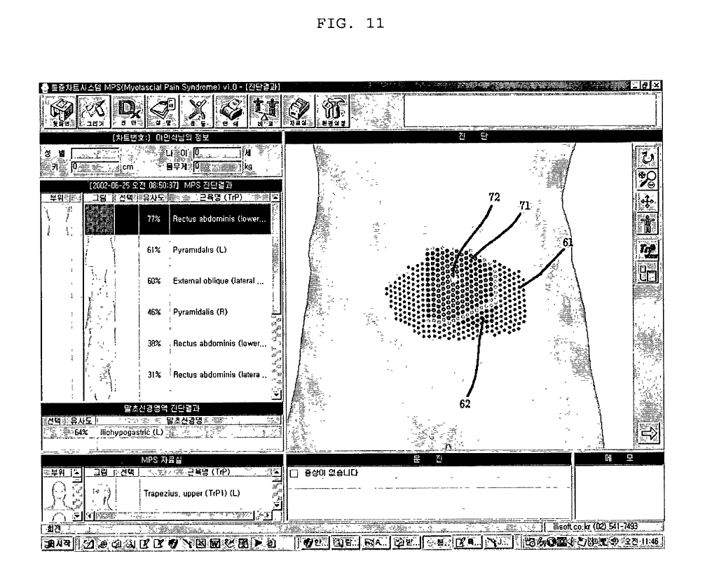

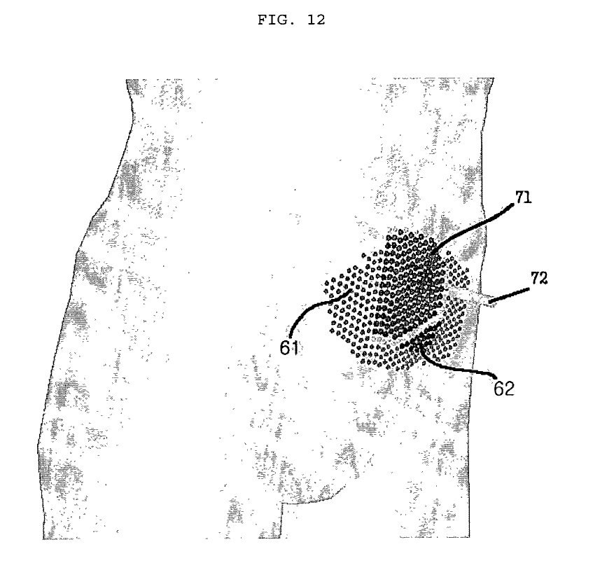

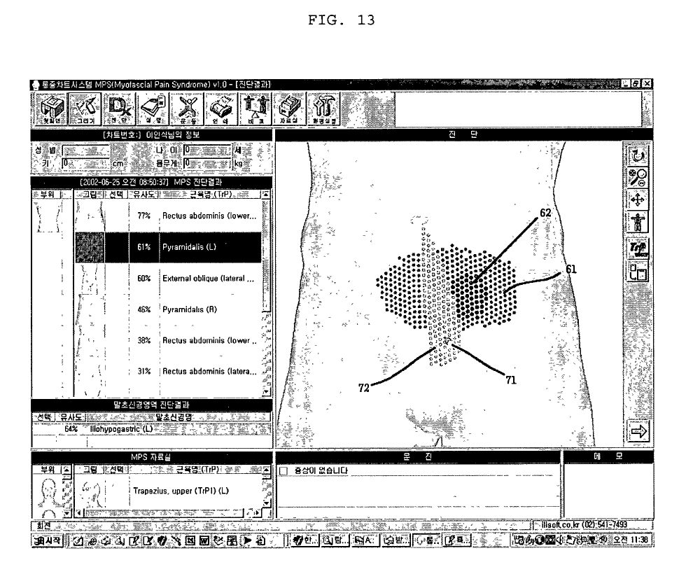

FIGS. 7 to 13 shows a screen provided by a program for which the system and method for pain diagnosis of the invention has been implemented as a system for diagnosis of myofascial pain syndromes (MPS). FIG. 7 shows a screen for entering pain areas, by a drawing method, on a three-dimensional human body model implemented in an input module of the system for MPS diagnosis. FIG. 8 shows a side view of the three-dimensional human body model shown in the right frame of FIG. 7. FIG. 9 shows a screen where pain areas have been inputted in a format of drawing after choosing a square labeled back and waist, among squares shown in the left frame of FIG. 7. FIG. 10 shows a screen for entering a patient's symptoms, in the format of a questionnaire, which is implemented in an input module of the system for MPS diagnosis. FIG. 11 shows a screen where diagnosis results have been derived by a diagnosis module after inputting pain areas on a three-dimensional human body model implemented in an input module. FIG. 12 shows a side view of a drawing represented on a three-dimensional human body model, upon choice of a first pain pattern (that is, Rectus abdominis (lower areas)) among pain patterns listed on the left frame of FIG. 11. FIG. 13 shows a screen of a drawing represented on a three-dimensional human body model, upon choice of a second pain pattern (that is, Pyramidalis (L)) among pain patterns listed on the left frame of FIG. 11

More particularly, FIG. 7 is a screen for entering pain areas on a three-dimensional human body model by a drawing method. The left frame shows squares for varying body parts (whole body, head and neck, shoulder and breast, back and waist, left arm, right arm, and legs). As one square is chosen among these squares, an enlarged drawing of a body part corresponding thereto appears on the right frame. In FIG. 7, the right frame shows a whole body upon choice of a square labeled whole body in the left frame.

Referring to FIGS. 7 to 9, a chosen body part may be reduced/enlarged, rotated, and translated for accurately inputting pain areas. The program may perform editing functions such as deletion and cancellation of execution request, sharpening, and filtering for noise removal, and color-coding according to degrees of pains.

For example, the three-dimensional human body model may be rotated and translated to enter pain areas in the side, as shown in FIG. 8.

FIG. 9 is a screen for drawing pain areas after choosing a square labeled back and waist among squares shown in the left frame of FIG. 7. FIG. 9 shows two distinguished sites of a painful site 61 and a severely painful site 62.

FIG. 10 shows a screen for entering patient's symptoms, in a format of a questionnaire, which is implemented in the input module, not for entering pain areas by a drawing method as in FIGS. 7 to 9. Possible symptoms corresponding to the selected pain patterns can be outputted, so the patient can check them. In FIG. 10, a list of questions corresponding to the first pain pattern (that is, Rectus abdominis (lower areas)) of FIG. 11 are outputted, including:

Does pain increase when bending over?

Do feet and legs feel hot?

Do you have cramps in the calf?

Do you have problems in making your legs comfortable during sleeping?

Through these ways, once patient's pain areas are inputted either in a method of drawing on a three-dimensional human body model or in the format of a questionnaire, MPS diagnosis results are derived through the diagnosis module. FIGS. 11 to 13 shows the diagnosis results.

As shown in FIG. 11, the first pain pattern 71 (Rectus abdominis (lower areas)) and its TrP 72, which are shown in a tree structure in the left frame, are represented on the three-dimensional human body model of the right frame, where the pain areas have been entered by the patient. In the left frame are represented referred muscles (including their TrPs) in a tree structure, according to the order of muscles in the pain areas where the patient expresses feelings of pain (a degree of similarity). Diagnosis results about peripheral nerves corresponding to the patient' pain areas are also represented.

FIG. 12 shows a side view of a drawing represented on a three-dimensional human body model, upon choice of a first pain pattern (that is, Rectus abdominis (lower areas)) among pain patterns listed on the left frame of FIG. 11. FIG. 13 shows a drawing represented on a three-dimensional human body model, upon choice of a second pain pattern (that is, Pyramidalis (L)) among pain patterns listed on the left frame of FIG. 11.

### Industrial Applicability

As apparent from the above description, the present invention provides a system for pain diagnosis and method therefor, enabling a patient to express painful sites and pain areas to a physician so as to communicate therebetween in efficient and accurate manners, and further enabling the physician to easily derive causes of pain and make a prescription therefor, thereby facilitating diagnosis and treatment in efficient and accurate manners.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A system for pain diagnosis, comprising:
an input module for receiving inputs of pain areas of a patient onto a three-dimensional human body model which comprises uniformly divided multiple cells;
a diagnosis module for deriving diagnosis results, comprising a database for storing data on various pain patterns, a submodule A for checking over a surface of the human body model according to divided multiple blocks, comparing the inputted pain areas with respect to the blocks, and deriving referred pain patterns with respect to the blocks corresponding to the patient's pain areas from the database, a submodule B for allowing the patient to confirm symptoms of the derived pain patterns and assigning weighted values to confirmed pain patterns, according to degrees of matching upon confirmation, a submodule C for comparing images of the confirmed pain patterns with the inputted pain areas, and a submodule D for calculating degrees of matching between the confirmed pain patterns and the inputted pain areas; and
an output module for outputting the diagnosis results derived from the diagnosis module.

2. The system as set forth in claim 1, wherein the input module includes a function of receiving inputs of pain symptoms of the patient in a format of answers to questions.

3. The system as set forth in claim 1, wherein the output module outputs the diagnosis results derived from the diagnosis module, in a tree structure according to the order of likelihood.

4. The system as set forth in claim 3, wherein the output module represents the pain patterns shown in the tree structure on a three-dimensional human body model where the patient's pain areas have been represented.

5. The system as set forth in claim 3, wherein the output module represents nerves shown in the tree structure on a three-dimensional human body model where the patient's pain areas have been represented.

6. The system as set forth in claim 1, wherein the output module concurrently outputs symptoms and a prescription corresponding to the diagnosis results.

7. A method for pain diagnosis, comprising the steps of: inputting patient's primary information and pain areas in an input module;
checking over a surface of a human body model according to divided multiple blocks, comparing the inputted pain areas with respect to the blocks, and deriving referred pain patterns with respect to the blocks corresponding to the patient's pain areas from the database, using a submodule A of a diagnosis module; allowing the patient to confirm symptoms of the derived pain patterns and assigning weighted values to confirmed pain patterns, according to degrees of matching upon confirmation, using a submodule B of the diagnosis module;
comparing images of the confirmed pain patterns with the inputted pain areas, using a submodule C of the diagnosis module; calculating degrees of matching between the confirmed pain patterns and the inputted pain areas, thereby deriving diagnosis results, using a submodule D of the diagnosis module; and
outputting the derived diagnosis results through an output module.
